(19) United States
(12) Patent Application Publication
(10) Pub. No.: US 2023/0381766 A1
(43) Pub. Date: Nov. 30, 2023

(54) STRUCTURED LAYER ARRANGEMENT AND METHOD FOR PRODUCING A LAYER ARRANGEMENT

(71) Applicant: DR. JOHANNES HEIDENHAIN GmbH, Traunreut (DE)

(72) Inventors: Peter SPECKBACHER, Kirchweidach (DE); Wolfgang SEDLMEIER, Obing (DE); Réka CSIKI, Traunstein (DE); Martina DOWY, Rosenheim (DE); Jenny OBERG, Traunstein (DE)

(21) Appl. No.: 18/031,726

(22) PCT Filed: Sep. 23, 2021
(86) PCT No.: PCT/EP2021/076140

§ 371 Date: Apr. 13, 2023

(30) Foreign Application Priority Data
Oct. 14, 2020 (DE) 10 2020 212 940.1

(51) Int. Cl. B01J 35/00; B01J 21/06; B01J 31/06; B01J 35/02; B01J 37/34

(52) U.S. Cl. CPC B01J 35/004; B01J 21/063; B01J 35/0006; B01J 31/063; B01J 35/023; B01J 37/34

(57) ABSTRACT

A structured layer arrangement includes a planar carrier substrate, on the functional-effective side of which a structured chromium layer is arranged. This includes chromium areas alternating with uncoated areas of the carrier substrate. Above the chromium layer, a two-dimensional reactive layer is arranged, which has a higher photocatalytic activity in partial areas above the chromium areas than in partial areas above the uncoated areas of the carrier substrate.

### FIELD OF THE INVENTION

The present invention relates to a structured layer arrangement and a method for producing such a layer arrangement.

### BACKGROUND INFORMATION

When examining material samples consisting of biological molecules by optical analysis methods, it is often necessary to structure the layered material samples, which are arranged on a carrier substrate, in a predetermined manner. This means that alternately arranged partial areas, in which the material samples are present or absent, must be created in a specific geometric form on the carrier substrate.

This can be accomplished, for example, by suitably modifying the carrier substrate locally. The corresponding biological molecules adhere in certain partial areas of the carrier substrate, while they are repelled in other partial areas. In this context, reference is made, for example, to the publication G. Panzarasa, G. Soliveri, Photocatalytic Lithography, Appl. Sci. 2019, 9, p. 1266. A disadvantage of such an approach is that the functional layers consist only of a single material which is modified by external influences such as light. This does not ensure long-term stability of the material sample.

Furthermore, it is possible to locally destroy an adhesive layer for the material sample which has been applied two-dimensionally to the carrier substrate. In this manner, non-adherent partial areas of the carrier substrate are exposed, in which no biological molecules of the material sample are present. Such a structuring variant is described, for example, in U.S. Patent Application Publication No. 2005/0266319 and includes depositing a mixture of a cell-binding material in the form of aminosilane and a binder material, e.g., photocatalytically active titanium oxide nanoparticles, on a carrier substrate in a homogeneous layer. This layer is exposed to UV radiation via a mask structure in a photolithography process, whereby the photocatalytic property of the binder material selectively destroys the cell-binding property of the aminosilane locally in predetermined partial areas. A disadvantage of this method is that it requires a complex mask-based photolithography process for structuring. Secondly, the binding areas also degrade in the long term.

### SUMMARY

Example embodiments of the present invention provide a structured layer arrangement, which is particularly suitable for optical analysis methods in biological and/or medical applications and which can be produced with as little cost as possible. Furthermore, a suitable production method for such a layer arrangement is provided.

According to an example embodiment of the present invention, a structured layer arrangement includes a planar carrier substrate and a structured chromium layer having chromium areas arranged alternately with uncoated areas of the carrier substrate on a functional-effective side of the carrier substrate. Above the structured chromium layer is a two-dimensional reactive layer, which has a higher photocatalytic activity in partial areas above the chromium areas than in partial areas above the uncoated areas of the carrier substrate.

For example, the reactive layer is formed of titanium oxide TiOₓ, with x=2-4; in this case, the partial areas with higher photocatalytic activity are formed predominantly of titanium oxide richer in anatase and the partial areas with lower photocatalytic activity are formed predominantly of titanium oxide richer in rutile.

It is possible that the reactive layer made of titanium oxide has a thickness in the range of 30 nm-300 nm.

The chromium layer can have a thickness in the range of 30 nm-150 nm.

Furthermore, the chromium layer can have a nitrogen content in the range of 15 at %-25 at %.

For example, the carrier substrate is formed of one of the following materials: glass; glass ceramic; and optically transparent crystal.

Furthermore, it can be provided that a biofunctional layer is arranged above the reactive layer.

In this regard, the biofunctional layer may be configured for specific binding or accumulation of biological molecules to the biofunctional layer.

For example, the biofunctional layer contains one or more of the following functional groups: Amino; Epoxy; Carboxyl; Hydroxyl; Thiol; and Azide.

Alternatively, it is also possible that the biofunctional layer is adapted to inhibit or prevent the binding or accumulation of biological molecules on the biofunctional layer.

For example, the biofunctional layer may include one or more of the following functional groups: PEG polymer; PEO polymer; HMDS; fluorine-terminated hydrocarbon chains; and saturated hydrocarbon chains.

Furthermore, it can be provided that the biofunctional layer includes a self-assembled monolayer, or an organosilane that forms an amorphous silicon oxide network to the reactive layer.

In this context, it is possible that the biofunctional layer is made of one of the following materials: 3-aminopropyltriethoxysilane (APTES); 3-aminopropyltrimethoxysilane (APTMS); N-(2-aminoethyl)-3-aminopropyltriethoxysilane (AEAPTES); N-(2-aminoethyl)-3-aminopropyltrimethoxysilane (AEAPTMS); N-(6-aminohexyl)aminomethyltriethoxysilane (AHAMTES); and 3-aminopropyldiisopropylethoxysilane (APDIPES).

It is further possible that a hexamethyldisilazane layer is arranged as a functional layer above the reactive layer.

Alternatively, it can be provided that a negative photoresist is arranged as a functional layer above the reactive layer.

It is further possible that a two-dimensional reflector layer, which is covered two-dimensionally by a dielectric layer, is arranged directly on the functional-effective side of the carrier substrate, and the structured chromium layer is arranged on the dielectric layer.

The reflector layer can be made of a metal and the dielectric layer can be made of silicon dioxide.

The method according to an example embodiment of the present invention for producing a structured layer arrangement includes: providing a planar carrier substrate; applying a structured chromium layer on the functional-effective side of the carrier substrate, which includes chromium areas arranged alternately with uncoated areas of the carrier substrate; and applying a two-dimensional reactive layer on the functional-effective side of the carrier substrate above the structured chromium layer, in which partial areas are formed in the reactive layer above the chromium areas with a higher photocatalytic activity than in the partial areas of the reactive layer above the uncoated areas of the carrier substrate.

Using a low-temperature sputtering process, a titanium oxide layer with a thickness in the range of 30 nm-300 nm is, for example, applied as the reactive layer.

An advantage of the structured layer arrangement and of the corresponding method described herein is that no complex, mask-based photolithography process is required to activate the photocatalysis in the desired partial areas. The structuring can be carried out in a simplified manner by illuminating the carrier substrate two-dimensionally with suitable electromagnetic radiation. This results in a structured layer arrangement that can be used in a variety of manners in biological and/or medical analytical systems with optical readout methods. Additionally, the biological molecules can be removed by a purification process using, for example, UV-ozone devices or purification plasma, whereby the structured layer arrangement is regenerated again and can thus be reused.

Further features and aspects of example embodiments of the present invention are explained in greater detail below with reference to the appended schematic Figures.

### BRIEF DESCRIPTION OF THE DRAWINGS

FIGS. 1a-1d illustrate a method for producing a structured layer arrangement.

FIG. 2 is a cross-sectional view of a structured layer arrangement.

FIGS. 3a-3c illustrate a method in connection with the application of a biofunctional layer on the structured layer arrangement.

FIG. 4 is a cross-sectional view of a structured layer arrangement.

FIGS. 5a-5e illustrate a method for producing a structured layer arrangement.

### DETAILED DESCRIPTION

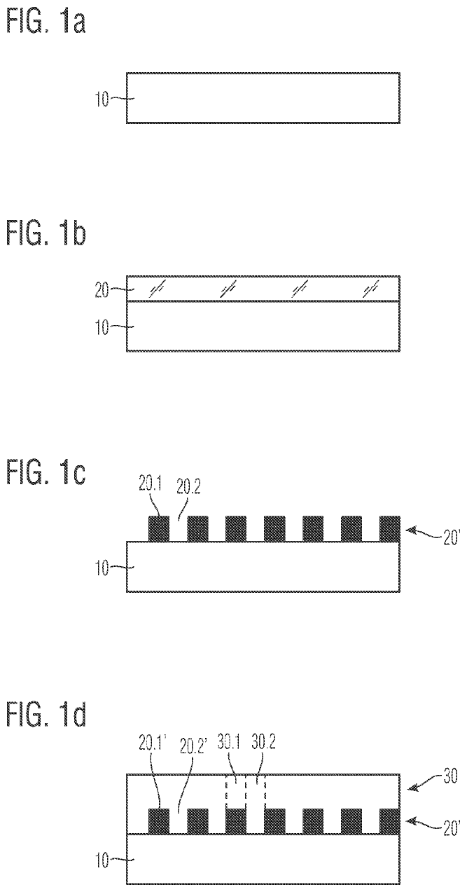

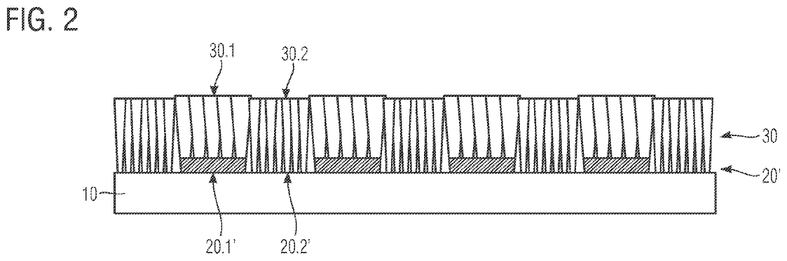

FIGS. 1a-1d illustrate a method for producing a structured layer arrangement according to the present invention are first explained below, and the structured layer arrangement is described in more detail with reference to FIG. 2.

First, as illustrated in FIG. 1a, a suitable plate-shaped or planar carrier substrate 10 is provided, which is, e.g., formed of glass. For example, the glass types D263, BF33 or quartz glass are considered suitable. Alternatively, it is possible to use glass ceramics such as Zerodur or suitable optically transparent crystals such as ZnSe or KBr as the material for the carrier substrate 10, which would have to be provided in a suitable plate-shaped or planar form. For example, the corresponding carrier substrate material has as low an autofluorescence as possible.

In the illustrated example, the build-up of the structured layer arrangement explained below takes place on the upward-facing side of the carrier substrate 10, which is also referred to as the functional-effective side of the carrier substrate 10. It should be understood that this does not represent any restriction with regard to the orientation of this side of the carrier substrate 10.

As illustrated in FIG. 1b, a chromium layer 20 is applied over the entire surface of the functional-effective side of the carrier substrate 10 with a thickness in the range of 30 nm-150 nm. For example, the chromium layer 20 has a nitrogen content in the range of 15 at %-25 at %, e.g., 18 at %-22 at %. For example, a sputtering method can be used to apply the chromium layer 20 to the carrier substrate 10.

Thereafter, the chromium layer 20 is lithographically structured. For this purpose, parts of the two-dimensional chromium layer 20 on the carrier substrate 10 are removed by a suitable lithography method, so that after this further method step, a structured chromium layer 20′ is present on the functional-effective side of the carrier substrate 10, as illustrated in FIG. 1c. The resulting structured chromium layer 20′ includes chromium areas 20.1′ arranged alternately with uncoated areas 20.2′ of the carrier substrate 10 on the functional-effective side of the carrier substrate 10.

Thereafter, as illustrated in FIG. 1d, a two-dimensional reactive layer 30 is applied to the functional-effective side of the carrier substrate 10 above the structured chromium layer 20′. In the present example, the material provided for the reactive layer 30 is titanium oxide TiOₓ, with x=2-4. For example, x is chosen in the range x=2.9-3.6 at the TiOₓ surface. The titanium oxide is deposited above the structured chromium layer 20′ via a low-temperature sputtering process with a thickness in the range of 30 nm-300 nm. Partial areas 30.1, 30.2, which have a different photocatalytic activity, are formed in the reactive layer 30. In this context, photocatalytic activity means that irradiation with electromagnetic radiation—such as light in a suitable wavelength range—can trigger a specific chemical reaction in the corresponding material.

Partial areas 30.1 of the reactive layer 30 are formed above the chromium areas 20.1′ with a higher photocatalytic activity than in the partial areas 30.2 of the reactive layer 30 above the uncoated areas 20.2′ of the carrier substrate 10. This is due to titanium oxide richer in anatase growing above the chromium areas 20.1 in the partial areas 30.1 of the reactive layer 30, which titanium oxide has a higher photocatalytic activity than the titanium oxide richer in rutile growing above the uncoated areas 20.2′ in the partial areas 30.2. The phase richer in anatase of the titanium oxide has a significantly higher photocatalytic activity than the phase richer in rutile of the titanium oxide. In the phase richer in anatase of the titanium oxide, a specific chemical reaction can be triggered by irradiation with light in a suitable wavelength range, as explained below.

An enlarged cross-sectional view of a structured layer arrangement is illustrated in FIG. 2. As illustrated, the titanium oxide richer in anatase grows on the chromium areas in the partial areas 30.1 of the reactive layer 30 in crystallites perpendicular to the carrier substrate surface. In the uncoated areas 20.2′ of the carrier substrate 10, on the other hand, the titanium oxide richer in rutile grows in the partial areas 30.2 of the reactive layer 30. In the partial areas 30.1 with the phase richer in anatase of the titanium oxide, somewhat larger crystallites are formed than in the partial areas 30.2 with the phase richer in rutile of the titanium oxide. Thus, in the partial areas 30.1 with the phase richer in anatase of the titanium oxide, a somewhat lower density of the reactive layer 30 is present than in the partial areas 30.2 with the phase richer in rutile of the titanium oxide.

During the growth of the reactive layer 30, fixed grain boundaries are formed in the boundary areas of adjacent partial areas 30.1, 30.2 between the two phases of the titanium oxide, via which grain boundaries the minimum structural widths of the reactive layer 30 are specified. At the surface of the reactive layer 30, transition areas result between the various partial areas with a lateral extension in the nanometer range, typically approximately a few 10 nanometers.

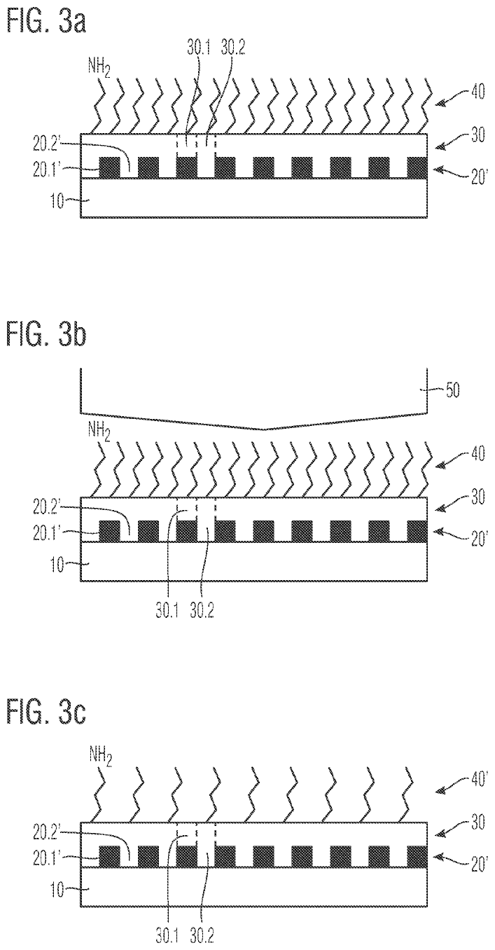

In this manner, alternating photocatalytic properties can be imparted to the layer arrangement, which are, for example, also stable over the long term. Referring to FIGS. 3a-3d, the following will explain how such a structured layer arrangement can be used to accumulate spatially structured biological molecules, such as proteins or nucleic acids, on it for analytical purposes.

FIG. 3a illustrates how a biofunctional layer 40 is first arranged above the reactive layer 30 on the layer arrangement as illustrated in FIG. 1d or FIG. 2. In the present example, the biofunctional layer 40 is configured for specific binding or accumulation of biological molecules to the free upper surface of the biofunctional layer 40. For example, it includes contains one or more of the following functional groups: Amino (—NH₂, NH₃, etc.); Epoxy; Carbox (—COOH); Hydroxyl (—OH); Thiol; and Azide.

The arrangement of such a biofunctional layer 40 is necessary because the reactive layer 30 formed from titanium oxide, although biocompatible due to its non-toxic properties, cannot form covalent bonds with biological molecules, but only adsorbed accumulations of suitable molecules.

In the present example, the biofunctional layer 40 includes an organosilane in the form of an aminosilane, which is deposited two-dimensionally on the layer arrangement above the reactive layer 30 via a suitable deposition method, such as a PECVD method or a desiccator method. During the deposition of aminosilanes, the alkyl groups are cleaved from the silicon atom so that the bond released at the silicon atom can bind to the substrate surface via an oxygen atom. This leads to the formation of an amorphous silicon oxide network through which the biofunctional layer 40 is stably bonded to the reactive layer 30 located thereunder.

Specifically, the materials listed below, for example, are considered well-suited as biofunctional layers 40: 3-aminopropyltriethoxysilane (APTES); 3-aminopropyltrimethoxysilane (APTMS); N-(2-aminoethyl)-3-aminopropyltriethoxysilane (AEAPTES); N-(2-aminoethyl)-3-aminopropyltrimethoxysilane (AEAPTMS); N-(6-aminohexyl)aminomethyltriethoxysilane (AHAMTES); and 3-aminopropyldiisopropylethoxysilane (APDIPES).

In the following method step, the functional-effective side of the layer arrangement with the biofunctional layer 40 arranged thereon is illuminated two-dimensionally with electromagnetic radiation 50. Illumination may be performed by a suitable UV light source in the ultraviolet wavelength range between 200 nm-400 nm, e.g., in the wavelength range of 350 nm-400 nm. Due to the irradiation, a bond-destroying effect results in the photocatalytically active partial areas 30.1 of the reactive layer 30 with titanium oxide richer in anatase. As a result of the photocatalysis, the organic bonds to the biofunctional layer 40 located thereabove break down in these partial areas 30.1 and the corresponding material of the biofunctional layer loses its binding ability locally. In the partial areas of the reactive layer 30 with the titanium oxide richer in rutile, due to the low photocatalytic activity, the UV irradiation does not result in any destruction of the bonds to the biofunctional layer 40 arranged thereabove.

After the corresponding irradiation, the structured arrangement of the biofunctional layer 40′ illustrated in FIG. 3c is present above the reactive layer 30. This can be used to form, for example, a structured array of biomolecules for analysis purposes, which can accumulate via the amino groups (—NH₂, NH₃+, etc.) in the remaining areas of the structured biofunctional layer 40′.

The biofunctional layer may also be adapted to inhibit or prevent thereabove the binding or accumulation of biological molecules on the biofunctional layer. For example, the biofunctional layer contains one or more of the following functional groups: PEG polymer; PEO polymer; HMDS; fluorine-terminated hydrocarbon chains; and saturated hydrocarbon chains.

Furthermore, it may also be provided that the biofunctional layer includes a self-assembled monolayer. This can include organophosphonates or organosilanes, with suitable binding or non-binding properties, respectively.

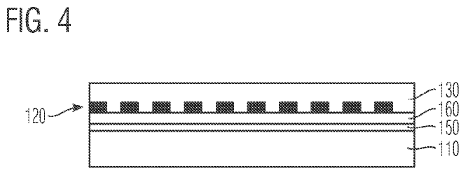

A further structured layer arrangement is illustrated in FIG. 4.

In this example, it is provided that a two-dimensional reflector layer 150, which is covered by a dielectric layer 160, is arranged directly on the functional-effective side of the carrier substrate 110. The reflector layer 150 in combination with a suitably selected dielectric layer thickness leads to a field increase in the area of the biomolecules, which ultimately results in a higher signal yield. As a result, the sensitivity of the optical readout method is increased. On the dielectric layer 160, first the structured chromium layer 120 is arranged, above which, as in the previous examples, the reactive layer 130 is arranged. Metals, such as aluminum or chromium, for example, may be utilized as materials for the reflector layer. For the dielectric layer, the use of silicon dioxide is possible. In an example embodiment with a fluorescence excitation wavelength of 490 nm, a reflector layer made of aluminum with a layer thickness in the range of 80 nm-100 nm is arranged on the carrier substrate 110. A dielectric layer 160 made of silicon dioxide is applied thereon, optionally with a layer thickness in the range of 10 nm-30 nm or 180 nm-200 nm. This is coated with a chromium layer having a thickness in the range of 30 nm-150 nm, which is covered with a 160 nm thick titanium oxide layer after the structuring.

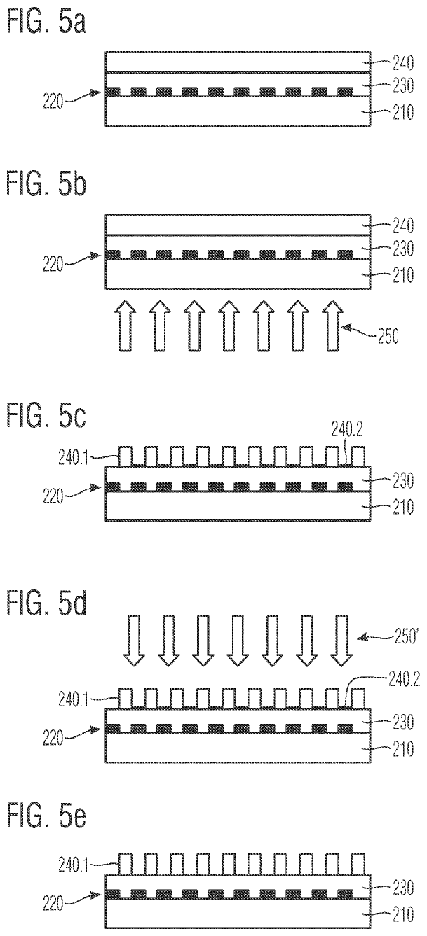

FIGS. 5a-5d illustrate a method for producing a further structured layer arrangement. Here, as the structurable functional layer 240, a negative photoresist is used, which is applied to the reactive layer 230 via a spin-on process as illustrated in FIG. 5a. As illustrated in FIG. 5b, electromagnetic irradiation in the ultraviolet range is applied to the surface from the direction of the carrier substrate 210, i.e., in the example illustrated, from below. As illustrated in FIG. 5c, in the partial areas of the carrier substrate 210 not coated with chromium, the negative resist 240.1 is completely retained in this case, while, after the development process, only small residues of the negative photoresist 240.2 remain on the partial areas of the reactive layer 230 which are richer in anatase. These residues of the negative photoresist 240.2 are completely removed by the further full-area irradiation with suitable ultraviolet radiation from the other direction, as illustrated in Figure so that, as illustrated in FIG. 5e, the layer arrangement with the desired structured reactive layer 240.1 remains, which can be used biofunctionally. Complete removal of the residues of the negative photoresist 240.2 reduces non-specific bonds of biomolecules and ensures effortless adhesion of biomolecules.

In another variant of the example embodiment illustrated in FIGS. 5a-5d, the negative photoresist used can be replaced by a thin metal or dielectric layer. This is structured using a suitable dry chemical method such that the gaps which become free are located above the partial areas richer in anatase. By irradiating from above with light of a wavelength of 200 nm-400 nm, e.g., 350 nm-400 nm, the remaining polymer residues resulting from the etching method are completely removed on the partial areas richer in anatase.

Instead of aminosilane or negative photoresist, other materials can also be used for layer modification. For example, a hexamethyldisilazane layer (HMDS layer) may also be used as a functional layer, which is deposited on the reactive layer via an evaporation method and irradiated two-dimensionally with electromagnetic radiation in the ultraviolet spectral range in the wavelength range of 200 nm-400 nm, e.g., 350 nm-400 nm. The property of the HMDS layer is modified by the photocatalysis in the partial areas of the reactive layer which are richer in anatase, while the property is retained in the partial areas which are richer in rutile.

1-18. (canceled)

19. A structured layer arrangement, comprising:
a planar carrier substrate;
a structured chromium layer including chromium areas arranged alternatingly with uncoated areas of the carrier substrate on a functional-effective side of the carrier substrate; and
a two-dimensional reactive layer arranged above the structured chromium layer and having a higher photocatalytic activity in partial areas above the chromium areas of the carrier substrate than in partial areas above the uncoated areas of the carrier substrate.

20. The structured layer arrangement according to claim 19, wherein the reactive layer is formed of titanium oxide TiOₓ, with x=2 to 4, and the partial areas with higher photocatalytic activity are formed predominantly of titanium oxide richer in anatase, and the partial areas with lower photocatalytic activity are formed predominantly of titanium oxide richer in rutile.

21. The structured layer arrangement according to claim 20, wherein the reactive layer made of titanium oxide has a thickness in the range of 30 nm to 300 nm.

22. The structured layer arrangement according to claim 19, wherein the chromium layer has a thickness in the range of 30 nm to 150 nm.

23. The structured layer arrangement according to claim 19, wherein the chromium layer has a nitrogen content in the range 15 at % to 25 at %.

24. The structured layer arrangement according to claim 19, wherein the carrier substrate is formed of glass, glass ceramic, and/or optically transparent crystal.

25. The structured layer arrangement according to claim 19, wherein a biofunctional layer is arranged above the reactive layer.

26. The structured layer arrangement according to claim 25, wherein the biofunctional layer is configured for specific binding or accumulation of biological molecules on the biofunctional layer.

27. The structured layer arrangement according to claim 26, wherein the biofunctional layer includes amino, epoxy, carboxyl, hydroxyl, thiol, and/or azide functional groups.

28. The structured layer arrangement according to claim 25, wherein the biofunctional layer is adapted to inhibit and/or prevent binding or accumulation of biological molecules on the biofunctional layer.

29. The structured layer arrangement according to claim 28, wherein the biofunctional layer includes a PEG polymer, a PEO polymer, HMDS, fluorine-terminated hydrocarbon chains, and/or saturated hydrocarbon chains.

30. The structured layer arrangement according to claim 26, wherein the biofunctional layer includes a self-assembled monolayer and an organosilane that forms an amorphous silicon oxide network to the reactive layer.

31. The structured layer arrangement according to claim 28, wherein the biofunctional layer includes a self-assembled monolayer or an organosilane that forms an amorphous silicon oxide network to the reactive layer.

32. The structured layer arrangement according to claim 27, wherein the biofunctional layer consists of 3-aminopropyltriethoxysilane (APTES), 3-aminopropyltrimethoxysilane (APTMS), N-(2-aminoethyl)-3-aminopropyltriethoxysilane (AEAPTES), N-(2-aminoethyl)-3-aminopropyltrimethoxysilane (AEAPTMS), N-(6-aminohexyl)aminomethyltriethoxysilane (AHAMTES), or 3-aminopropyldiisopropylethoxysilane (APDIPES).

33. The structured layer arrangement according to claim 19, wherein a negative photoresist is arranged as a functional layer above the reactive layer.

34. The structured layer arrangement according to claim 19, wherein a two-dimensional reflector layer, covered two-dimensionally by a dielectric layer, is arranged directly on the functional-effective side of the carrier substrate, and the structured chromium layer is arranged on the dielectric layer.

35. The structured layer arrangement according to claim 34, wherein the reflector layer includes a metal, and the dielectric layer includes silicon dioxide.

36. A method for producing a structured layer arrangement, comprising:
applying a structured chromium layer on a functional-effective side of a planar carrier substrate, the structured chromium layer including chromium areas arranged alternatingly with uncoated areas of the carrier substrate;
applying a two-dimensional reactive layer on the functional-effective side of the carrier substrate above the structured chromium layer, partial areas of the reactive layer above the chromium areas having a higher photocatalytic activity than in partial areas of the reactive layer above the uncoated areas of the carrier substrate.

37. The method according to claim 36, wherein a titanium oxide layer is applied as the reactive layer via a low-temperature sputtering process with a thickness in the range of 30 nm to 300 nm.

38. A method for producing a structured layer arrangement as recited in claim 19, comprising:
applying a structured chromium layer on a functional-effective side of a planar carrier substrate, the structured chromium layer including chromium areas arranged alternatingly with uncoated areas of the carrier substrate;
applying a two-dimensional reactive layer on the functional-effective side of the carrier substrate above the structured chromium layer, partial areas of the reactive layer above the chromium areas having a higher photocatalytic activity than in partial areas of the reactive layer above the uncoated areas of the carrier substrate.

39. A structured layer arrangement, comprising:
a planar carrier substrate;
a structured chromium layer including chromium areas arranged alternatingly with uncoated areas of the carrier substrate on a functional-effective side of the carrier substrate; and
a two-dimensional reactive layer arranged above the structured chromium layer and having a higher photocatalytic activity in partial areas above the chromium areas of the carrier substrate than in partial areas above the uncoated areas of the carrier substrate;
wherein the structured layer arrangement is produced according to the method recited in claim 36.
